(19) United States
(12) Patent Application Publication
(10) Pub. No.: US 2016/0151187 A1
(43) Pub. Date: Jun. 2, 2016

(54) WALKING APPARATUS WITH A MULTIPLE BLADDER SYSTEM

(71) Applicant: Ovation Medical, Agoura Hills, CA (US)

(72) Inventor: Tracy E. GRIM, Thousand Oaks, CA (US)

(21) Appl. No.: 14/956,204

(22) Filed: Dec. 1, 2015

Related U.S. Application Data
(60) Provisional application No. 62/086,624, filed on Dec. 2, 2014.

(51) Int. Cl. A61F 5/01

(52) U.S. Cl. CPC A61F 5/0111; A61F 5/0195

(57) ABSTRACT

A walking apparatus is provided that includes a frame, and a bladder system operatively coupled to the frame, the bladder system including a first displacement pump, a first bladder operatively coupled tot eh first displacement pump, a second displacement pump, and a second bladder operatively coupled to the second displacement pump.

### BACKGROUND

1. Field

The present disclosure relates generally to orthopedic walking boots.

2. Background

It is common that people, especially active and/or frail people, experience a variety of lower leg and ankle injuries. To aid in the treatment of the injuries it is desirable to immobilize the injury, typically above and below the affected joint.

Physicians traditionally place a patient's leg in a short leg cast, which is a cast that usually begins at the patient's toes and ends below the patient's knee. Generally, casts retain heat, cause an itching sensation on the skin, and rub against the leg particularly after swelling of the leg subsides.

An alternative to the short leg cast is an orthopedic walking boot, or a premanufactured orthopedic walking boot, that is made of a rigid plastic frame lined with a soft component (e.g, a soft padding or a soft good) to accommodate the leg comfortably. The orthopedic walking boots can be removed to treat skin problems, such as, to remove sutures or conduct passive range of motion exercises. Short leg casts do not offer the luxury of easy on/off, and the cost associated with applying another cast after removal.

An orthopedic walking boot is primarily a rigid encasing that usually envelopes the leg and immobilizes the foot and ankle at a neutral position (e.g., the foot extends 90 degrees relative to the leg). The patient can walk easiest if the ankle is fixed at 90 degrees. At angles other than 90 degrees the patient will be walking on the toes or on the heel thereby altering the gait pattern of the patient. The outer sole of the foot is generally curved from front to back in a rocker bottom fashion. The curvature of the outer sole provides a smoother stride from front to back allowing the heel to strike the ground first, followed by a rocking of foot forward, and finally a push off on the toes for a successful step.

### SUMMARY

In accordance with certain aspects of the present disclosure a walking apparatus is provided that includes a frame, and a bladder system operatively coupled to the frame, the bladder system including a first displacement pump, a first bladder operatively coupled tot eh first displacement pump, a second displacement pump, and a second bladder operatively coupled to the second displacement pump.

### BRIEF DESCRIPTION OF THE DRAWINGS

FIG. 1 illustrates an exploded perspective view of a walking apparatus in accordance with certain aspects of the present disclosure;

FIG. 1A illustrates a perspective view of a walking apparatus in accordance with certain aspects of the present disclosure;

FIG. 1B illustrates a perspective view of a bladder system in accordance with certain aspects of the present disclosure;

FIG. 2 illustrates a perspective view of a bladder system in accordance with certain aspects of the present disclosure; and

FIG. 3 illustrates a perspective view of a cam for use in a bladder system in accordance with certain aspects of the present disclosure.

### DETAILED DESCRIPTION

The detailed description set forth below in connection with the appended drawings is intended as a description of various aspects of the present invention and is not intended to represent all aspects in which the present invention may be practiced. The detailed description includes specific details for the purpose of providing a thorough understanding of the present invention. However, it will be apparent to those skilled in the art that the present invention may be practiced without these specific details. In some instances, well-known structures and components are shown in block diagram form in order to avoid obscuring the concepts of the present disclosure.

Various aspects of the present disclosure may provide a walking apparatus with a multiple bladder system that can be fitted around the leg to provide support and/or therapy, which can allow ambulation for an affected limb.

People often experience injuries to the lower leg and ankle. For example, blunt trauma, sports injuries and common falls are the primary causes of injuries of the lower leg. Injuries such as fractures of the bones or soft tissue injuries (e.g., ligamentous tears) have similar symptoms. Swelling, pain and inability to ambulate without support are expected and predictable. Some injuries may need to be immobilized for a period of time to facilitate healing. The time required for ligamentous injuries to heal is similar to the time required for fractures to heal, e.g., period of 4 to 6 weeks of immobilization is common. Different injuries require different rehab times and regimes.

Aspects of the present disclosure are directed to a walking apparatus with a multiple bladder system to selectively apply a hot or cold therapy and/or a selected pressure to an injured lower leg and/or ankle. In an aspect of the prevention disclosure, the walking apparatus can include an orthopedic walking boot with bilateral struts which connect a base of the orthopedic walking boot to an upper portion of the orthopedic walking boot. The struts may be rigid and provided on either side of the leg. The bilateral struts may be held onto the limb with strapping systems that encircle at least a portion of the limb. In another aspect, the base may be attached to a posterior piece which extends from the foot to the back of the leg and calf forming a clamshell configuration. In the clamshell configuration, a single piece encompasses a portion of the side of the leg (similar to the bilateral configuration) as well as the rear of the leg. The orthopedic walking boot may include an adjoining anterior piece that joins or overlaps the posterior piece and is held on by a traditional strapping system or with one or more mechanical attachment mechanisms. In another aspect, the orthopedic walking boot may comprise a “hybrid” configuration (also referred herein as a “multi-sectioned” configuration). In the hybrid configuration, the base may be attached to the bilateral struts of the bilateral configuration and also attached a separate/non-integral posterior element that encompasses the rear of leg (similar to the rear portion of the clamshell). In this manner, the bilateral struts surround the side of the legs while the separate posterior portion encompasses the rear of the leg. Thus, the hybrid configuration achieves a similar result as the clamshell with multiple sections, hence, “multi-sectioned.”

According to one aspect of the present disclosure, the orthopedic walking boot may be configured such that the portion that receives the user's foot (e.g., the base portion) extends at a 90° angle or at substantially 90° relative to a longitudinal axis of the portion that receives the user's leg (e.g., the upper portion). In another aspect, the orthopedic walking boot may include two struts rising from the base. The orthopedic walking boot may further include a soft component within the constraints of the struts or the clamshell, and on top of the base. The soft component may be held by straps.

Furthermore, the orthopedic walking boot may be configured to accommodate a soft component that can contain a multiple bladder system, in accordance with one aspect of the present disclosure. The multiple bladder system can include one or more individual bladders that can be filled and/or inflated with a liquid and/or air. For example, at least one of the bladders in the system can be inflated with a liquid and at least one of the other bladders in the system can be inflated with air. In such an instance, the bladder that is configured to be filled with liquid can be oriented within the bladder system to be closest to the skin of the patient to effectively apply a hot and/or cold therapy thereto. The bladder configured for inflation with air can be oriented behind the liquid bladder such that when the air bladder is inflated, the liquid bladder is pressed securely against the injured region to apply the hot and/or cold therapy to the injured region, while at the same time the added pressure provided by the inflated air bladder can provide additional support to the lower leg. Consequently, the adjustable walking apparatus of the present disclosure may be able to provide a better fit and support.

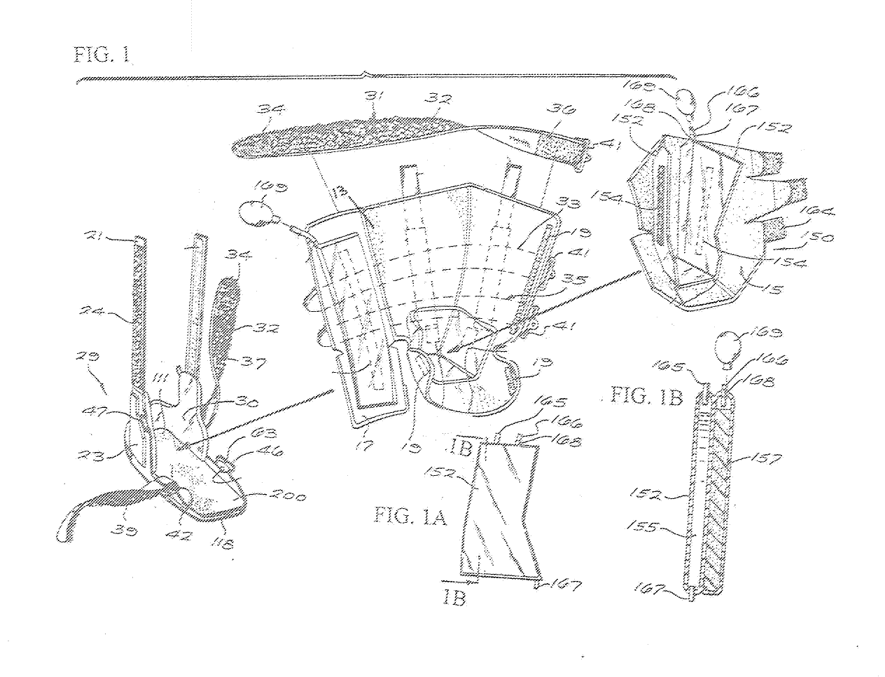

FIG. 1 depicts an exploded view of a walking apparatus 11 that can include a durable, resilient and removable foam and fabric outer covering soft goods-type support which may be formed of foam material which is substantially covered with a VELCRO™ hook receivable material such as brushed nylon. The soft goods-type support can include a tongue member 17 also covered with a VELCRO™ hook receivable material 15. While the soft component 13 is in an open position as shown in FIG. 1 it is able to receive an injured lower leg extremity. Additionally, the soft component 13 can receive an injured lower leg extremity which has been stabilized and supported by an additional ankle support 150.

The soft component 13, with its VELCRO™ hook receivable material, can extend around the injured limb below the knee while leaving the toes exposed, and the two exposed edges are securely held together by an inner portion of the soft component covered with a strip of VELCRO™ hook material 19, extending along the inner edges of the soft component 13. The walking apparatus 11 can include a rigid frame 29 including of a pair of struts 21, strut bases 23, and a rigid full-length sole 25, or a clamshell support (not shown). The frame 29 may be constructed of any rigid, lightweight material; however, it is preferred that a glass fiber reinforced nylon be used because it is relatively inexpensive, lightweight and sturdy.

The soft component 13 can be received between a pair of rigid struts which may be rigid or semi-rigid. The struts 21 can be firmly attached to a pair of strut bases 23. The strut bases 23 can also be integrally molded to a rigid, full-length sole, and the bottom of the soft component 13 can rests against the top of the sole covered with a custom molded innersole cushion 200. The heel portion 111 of the soft component 13 can be adjacent to a raised heel strip (not show) which is preferably made of a rigid material. A raised heel strip (not shown) can extend along the outer perimeter of the heel portion 111 of the sole, within strut bases 23, and its height is approximately 2 inches from the upper surface of the sole. A resilient support member 30 which may be a bladder inflated with air and/or liquid or lined with any type of resilient material such as a gel or foam, can cover the inner surfaces of the raised heel strip 43 and strut bases 23 and also may extend a substantial distance along the inner surface of each strut 21. Alternately, the soft component 13 can be received within a clamshell support member.

A resilient support member 30 can provide additional support around the heel and ankle areas of the patient's foot while he or she is wearing the walking apparatus 29. In addition, or alternatively, the inside surface of the soft component 13 surrounding the heel and ankle of the patient may be provided with a similar resilient support member that is spaced by the foam material of support 13 from the raised heel strip 43 and strut bases 23 and struts 21.

A plurality of straps 31, 33, 35, 37 and 39 can be used to affix the foot and the lower leg (within ankle support 150 and soft component 13) to the walking apparatus 29 and the struts 21, substantially eliminating any substantially detrimental flexion of the ankle or foot with respect to the leg. The struts 21 can be slightly flexible at the top in order to accommodate a range of leg sizes. Structurally, the struts 21 can act as cantilever beams that are subjected to flexure and torsion loads and are stabilized by their being affixed to the leg. Straps 31, 33, 35 each can have a D-loop 41 attached at one end. Straps 31, 33, 35 can be substantially covered with a VELCRO™ hook receivable material 32 on one side with a relatively small portion of VELCRO™ hook fabric 34 on the same side, opposite the D-loop 41. The opposite side of the straps 31, 33, 35 can be partially covered with a VELCRO™ hook fabric 36 extending from the end attached to the D-loop 41. The straps 31, 33, 35 can be used to secure the leg to the struts 21 by attaching the side of the straps 31, 33, 35 partially covered with the VELCRO™ hook fabric 36 to the soft component 13 or to a portion of the struts 21 covered with VELCRO™ hook receivable material 24 and passing the loose end of each strap 31, 33, 35, without the D-loop 41, through the D-loop 41 and tightly securing the small portion of VELCRO™ hook fabric 34 to the portion of the straps 31, 33, 35 substantially covered with VELCRO™ hook receivable fabric 32.

Strap 37 can extend across the dorsum of the instep, and is used to pull the encased foot down against the sole and back against the portion of the heel 111 of the sole consisting of a raised, resilient heel strip (not shown). This strap 37 can be constructed of an elastic fabric covered on one side with mostly VELCRO™ hook receivable fabric 32 and a small portion on the loose end with VELCRO™ hook fabric material 34. This strap 37 is securely connected to an integrally molded slot 45 located on one of the strut bases 23 and passes through a second integrally molded slot 47, and can be folded over itself to adjustably and securely attach the VELCRO™ hook fabric end 34 with the VELCRO™ hook receivable portion 32. The fastening of this strap 37 results in stabilization of the foot against upward, rearward and forward motion.

FIG. 1 further illustrates a forward-most strap 39 which can be used to hold the forefoot in a fixed or semi-fixed position. The VELCRO™ arrangements for the forward-most strap 39 can be similar to the arrangements on the elastic strap 37. The forward-most strap 39 can be securely and permanently affixed to a loop 42 which is, in turn, securely affixed to a U-shaped member. The U-shaped member can be constructed from a high-strength plastic and be ultrasonically welded to the sole through a bore hole (not shown) formed on the side of the sole.

On the opposite side of the sole a similar arrangement exists wherein a loop 46 is secured to a second U-shaped member 63 which can be ultrasonically welded through a second bore hole on the side of the sole, except that the strap 39 may not be permanently affixed to this loop 46. Subsequently, the loose end of the strap 39 passes through the loop 46 in order to have the VELCRO™ portions fold back over and adjustably attach to one another.

The strut 21 illustrated in FIG. 1 can be mated and securely attached to the strut base 23 by being inserted into a recess (not shown) on the inner portion of the struts base 23 which can be molded to receive the bottom portion of the struts 21. An adhesive may be used to secure the strut 21 to the strut base 23 into its recess 51. Metal pins (not shown) can be used to further reinforce the connection of the struts 21 and their respective strut bases 23.

As shown in FIG. 1, the manner in which the injured limb can be supported by walking apparatus 11 can be by inserting the foot and lower leg extremity into a resilient and adjustable lightweight soft component 13. The soft component 13 can be comprised of a soft goods-type of material, such as a SPANDEX-covered tricot, foam, or neoprene, and is able to receive a wide range of leg and foot sizes. The height of the soft component 13 can extend a distance to a point preferably above the ankle. A thickness of the soft component 13 can be in a range of about 1/16″ to ⅜″ thick. Furthermore, the soft component 13 can also covered with a VELCRO™ hook receivable material 15, i.e., brushed nylon. A pair of bladders 152 which may be inflated with air or filled with fluid, can be mounted on the inner side walls of the soft component 13 by means of a VELCRO™ hook fabric 154 that covers the surface of the bladders 152 attached to the inner surface of the soft component 13 and juxtaposed to the injured limb or joint. Although not shown, the bladders can alternately be received within pockets formed on an inner surface of the soft component 13. In addition, the soft component 13 can include a plurality of VELCRO™ hook fabric straps 164 which can be tightly wrapped around the injured limb and connected to the outer VELCRO™ hook receivable covering 15 of the soft component 13. The outer side walls of the soft component 13 can each include a pocket or pouch 158. These pockets 158 can be formed substantially long enough to receive a splint material 160, which can prevent any substantial flexion that may hinder the proper and rapid healing of the ankle. For example, a pocket 158 can be provided on the outer left side and on the outer right side of the soft component 150. A molded, rigid lightweight splint material 160, such as plastic, is fully received into each pocket 158 and closed off by a VELCRO™ hook fabric flap 162 which is located on the uppermost open end of each pocket 158 to prevent the splints 160 from leaving their respective pockets.

While the lower leg extremity is supported by the soft component 150, additional support can be provided by the inflatable bladders 152. Any type of resilient material may be used to form the bladders 152 that can house the liquid and/or air without leaking.

Either before or after the foot is enclosed within the soft component 150, the bladders 152 can be inflated with air and/or filled with a liquid until a comfortable, yet trim fit results around the injured limb. The bladders 152 can be inflated by a hollow tube 166 securely inserted into a port opening 168 in the bladder. Subsequently, a hollow resilient pump bulb 169 can be attached to the open end of the hollow tube 166 and squeezed a number of times to achieve the desired amount of inflation and support. Alternately, a pump bulb (not shown) can be built into the soft-goods type component 150 so that the user is not required to attach a bulb to the hollow tube to inflate the air bladder. The soft component 13 can include two inflatable bladder chambers 152 separated by seams 180 to prevent inflation pressure along the sole and heel of the foot. The bladders 152 can be constructed of a resilient material, such as an elastane or a polymer with a urethane laminate coating on the inside therein. A urethane bladder with tricot material facing the ankle could also be used, for example.

The bladders 152 may be formed of a smooth knit material such as a fabric including nylon and/or LYCRA on the outside, and a thin layer or film of plastic material such as urethane, on the inside. For example, a bladder laminate can include a fabric about 15 millimeters thick and a urethane film about 1½ millimeters thick, with the film being bonded to the fabric by an adhesive such as an ethyl vinyl alcohol adhesive having a copolymer resin base. Of course the materials and thicknesses of the bladders 152 can be varied without departing from the scope of the present disclosure.

FIGS. 1A and 1B illustrate an alternative embodiment with regard to the design of the bladders 152 which can be mounted within the soft component 13. In this embodiment, the bladder system is constructed of three layers of plastic welded to form one unit consisting of two separate chambers 155, 157. The first chamber can lie adjacent to the patient's skin and can receive hot or cold fluids for therapy. Hot/cold therapy can be advantageous in the healing process since it can reduce swelling of the injured limb and the degree in which the injured limb may atrophy by promoting improved blood circulation throughout the lower leg. The first chamber 155 can include a fluid entrance port 165 located at the top of the first chamber 155. Although the fluid entrance port 165 is illustrated as being located at the top of the first chamber 155, it is understood that the fluid entrance port can be located anywhere on the first chamber 155 to facilitate ease of use without departing from the scope of the present disclosure. The fluid entrance port 165 can allow for a fluid to be supplied throughout the first chamber 155. Any number of fluids may be supplied into the first chamber 155, for example, hot and/or cold water. Located at the bottom portion of the first chamber 155 can be an exit valve with a drain plug or clamp 167. The exit valve 167 can allow the patient to drain and remove the fluid from the first chamber 155. The second chamber 157 can support and stabilize the injured ankle of the patient by adjustably inflating the second chamber 157 with air or another gas. The second chamber can include a hollow air input tube 166 which is located at the top of the second chamber 157 and is securely inserted into a port opening 168 at the top of the second chamber 157. Subsequently, a hollow resilient pump bulb 169 can be attached to the open end of the hollow air input tube 166 and be squeezed a number of times to achieve the desired amount of inflation and support. Alternately, the pump bulb 169 can be operatively coupled to the air input tube 166 and built into the soft component 13 during manufacture, such that the user can inflate the bladder by depressing the pump bulb without the need to connect the pump bulb to the hollow air input tube 166. The second chamber 157 is provided with a valve arrangement for retaining air in the second chamber 157 and for selective deflation. Furthermore, when not inflated, the second chamber 157 may have a resilient material (for example, foam) inside it for additional cushioning.

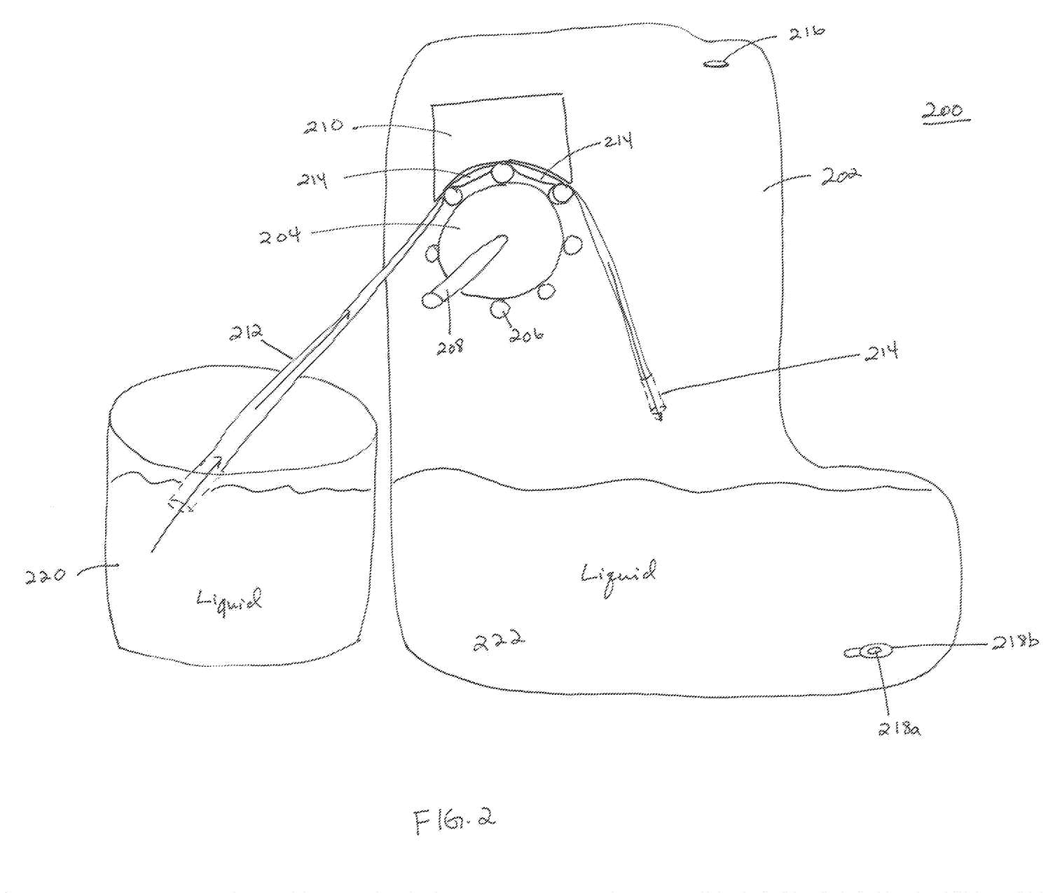

FIG. 2 illustrates a bladder system 200 according to one aspect of the present disclosure in which a bladder 202 is configured to receive a hot or cold liquid 220, 222 which can help alleviate pain and/or swelling of an injured ankle or lower leg. The bladder 202 may be made of an elastomeric material, a polymeric material, or a latex material just to name a few. The bladder system 200 in FIG. 2 is depicted as including a peristaltic-type pump that can transport the liquid 220, 222 into and/or out of the bladder 202. As seen in FIG. 2, components of the peristaltic-type pump can include a stator 210 and a cam 204. For example, the cam 204 may include a rotor that can include a plurality of occlusion elements 206. A tubing member 212 can be fitted between the stator 210 and cam 204, and may include a portion 212 that is external to the bladder 202 and a portion 214 that is located within the internal cavity of the bladder. Alternatively, the tubing member 212 can form a seal around an input port on the bladder 202 and not extend into the interior of the bladder 202. Furthermore, the occlusion elements 206 of the cam 204 can be configured to squeeze the tubing member 204 over a section 224 containing the liquid 220, such that the squeezed fluid in section 224 can move progressively around the tubing member 212 under the effect of a rotation of the cam 204. The rotation of the cam 204 can be effected manually or automatically, and an actuator 208 can be attached to the cam 204 and used to effect manual rotation of the cam 204. A motor (not shown) can also be connected to the cam 204 and used to automatically effect rotation. As the bladder 202 is filled with the liquid 222, air within the bladder can escape using an exit hole 216 formed near the top of the bladder 202. A drain hole 218a can be located near the bottom of the bladder 202 so that the liquid within the bladder 202 can be drained. A plugging member 218b can be received within the drain hole 218a to prevent liquid from leaking out of the bladder 202 during use.

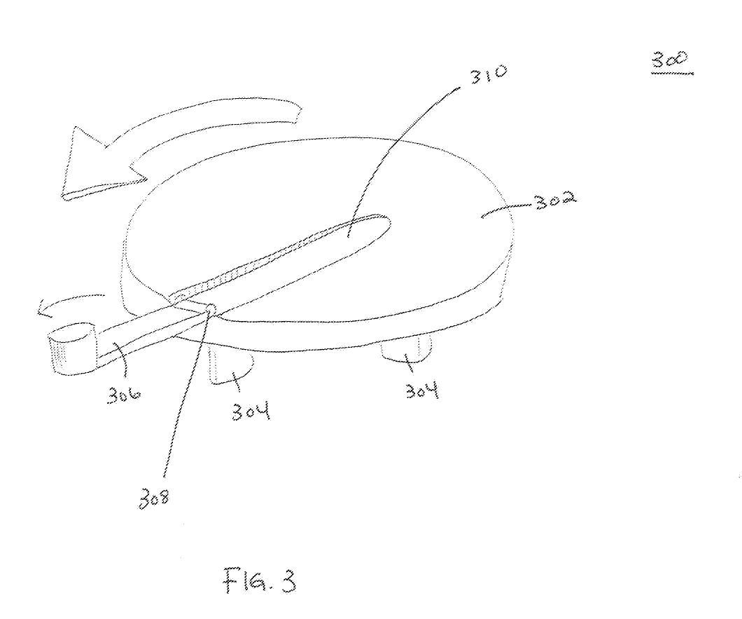

FIG. 3 depicts a cam that can be used in a bladder system of a walking apparatus according to certain aspects of the present disclosure. As seen in FIG. 3, the cam 300 can include a rotor body 302 that includes a cavity 310 on a surface thereof that is configured to receive the actuator 306 when the actuator is not in use. A hinge 308 can be used to operatively couple the actuator 306 to the rotor body 302, which can allow the actuator 306 to fold out for manual rotation and to be folded back into the cavity 310 when not in use. The cavity 310 on the surface of the rotor body 302 can be configured such that when the actuator 306 is nested therein, a substantially unitary surface with the rotor body is formed. Furthermore, the actuator 306 can be configured to manually rotate the cam 300, including occlusion elements 304, when a predetermined force is applied by a user. The rotational motion of the cam 300 can allow liquid to be moved into or out of the bladder 202 as depicted in FIG. 2.

It is understood, that any of the aspects illustrated, depicted, or described in the present disclosure, including in the figures, can be combined and/or used with any other aspect illustrated, depicted, or described in the present disclosure, including the figures. For example, each of the aspects set forth in the figures and/or described in the disclosure can be used in or combined with an open-toe orthopedic walking boot, a closed-toe orthopedic walking boot, an orthopedic walking boot including bilateral struts, an orthopedic walking boot including a clamshell configuration, a soft component of an orthopedic walking boot, a post-operative shoe (e.g., a shoe with a stiff base that can be worn on either the left foot or the right foot that can enable a patient to walk without reinjuring the toe(s) and/or foot), a clinical walker, and a hospital walker, just to name a few. Each of the bladders described above and illustrated in the figures can be combined with any variety of pumping mechanisms without departing from the scope of the present disclosure. For example, the pumping mechanisms that may be used in bladder system of the present disclosure may include a peristaltic pump, a pneumatic pump, a vacuum pump, a partial vacuum pump, a displacement pump, a lobe pump, a toepler pump, and a piston pump just to name a few.

Moreover, the claims are not intended to be limited to the various aspects of this disclosure, but are to be accorded the full scope consistent with the language of the claims. It is noted that specific illustrative embodiments of the disclosure have been shown in the drawings and described in detail hereinabove. It is to be understood that various changes and modifications may be made without departing from the spirit and scope of the disclosure. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.”

What is claimed is:

1. A walking apparatus, comprising:
a frame; and
a bladder system operatively coupled to the frame, the bladder system comprising:
a first displacement pump;
a first bladder operatively coupled to the first displacement pump;
a second displacement pump; and
a second bladder operatively coupled to the second displacement pump.

2. The apparatus of claim 1, wherein the first displacement pump is configured to provide air to the first bladder.

3. The apparatus of claim 2, wherein the second displacement pump is configured to provide liquid to the second bladder.

4. The apparatus of claim 1, wherein the first displacement pump includes:
a tubing member operatively coupled to the first bladder, the tubing member including an inlet port that is external to the first bladder and an outlet port that opens into an interior of the first bladder; and
a bulb operatively coupled to the tubing member, wherein the bulb is external to the first bladder.

5. The apparatus of claim 4, wherein the bulb is configured for actuation.

6. The apparatus of claim 5, wherein the bulb is configured to create a partial vacuum that moves gas molecules through the bulb and into the first bladder when the bulb is actuated.

7. The apparatus of claim 4, wherein the first displacement pump further includes a release valve configured to release of gas molecules from within the first bladder.

8. The apparatus of claim 4, wherein the second displacement pump includes:
a tubing member operatively coupled to the second bladder, the tubing member including an inlet port that is external to the second bladder and an outlet port that opens into an interior of the second bladder;
a stator;
a cam operatively coupled to the tubing member, the cam including at least one occlusion member; and
an actuator operatively coupled to the cam.

9. The apparatus of claim 8, wherein when the actuator is actuated, the cam is configured to rotate and the at least one occlusion member is configured to occlude a portion of the tubing member between the cam and the stator forcing a liquid through the tubing member and into the second bladder.

10. The apparatus of claim 8, wherein the actuator includes a manual actuator configured for manual rotation.

11. The apparatus of claim 10, wherein:
the manual actuator is operatively coupled to the cam using an hinge member; and
the manual actuator is configured to be received within a cavity on a surface of the cam when the actuator is not in use.

12. The apparatus of claim 8, wherein the actuator includes a motor configured for automatic rotation.

13. The apparatus of claim 3, wherein the second bladder includes a first aperture configured to enable an escape of air when the second bladder is filled with the liquid.

14. The apparatus of claim 3, wherein the second bladder further includes a second aperture configured to enable an escape of liquid during an emptying of the second bladder.

15. The apparatus of claim 14, wherein:
the bladder system further comprises a plugging member; and
the second aperture is configured to receive the plugging member.

16. The apparatus of claim 3, wherein the second bladder is configured for positioning adjacent to a skin layer.

17. The apparatus of claim 3, wherein the first bladder shares a surface with the second bladder.

18. The apparatus of claim 3, wherein the first bladder and the second bladder are separate.

19. The apparatus of claim 8, further comprising a padding component operatively coupled to the frame, wherein the first displacement pump and the second displacement pump are operatively coupled to the padding component.

20. The apparatus of claim 19, wherein the first bladder and the second bladder are located within the padding component.

21. The apparatus of claim 19, wherein the padding component includes at least one pocket configured to receive each of the first bladder and the second bladder.

22. The apparatus of claim 1, wherein the walking apparatus comprises an open-toe orthopedic walking boot, a closed-toe orthopedic walking boot, an orthopedic walking boot including bilateral struts, an orthopedic walking boot including a clamshell configuration, a soft component of an orthopedic walking boot, a post-operative shoe, a clinical walker, or a hospital walker.
